(19) United States
(12) Patent Application Publication
(10) Pub. No.: US 2021/0125701 A1
(43) Pub. Date: Apr. 29, 2021

(54) System, Method, Process and Apparatus for Assisting in Formulating and Attaining Healthy Weight Management Goals

(71) Applicant: One Number, LLC, Fayetteville, TX (US)

(72) Inventors: Reagan D. Brown, Fayetteville, TX (US); Tom H. Hill, Fayetteville, TX (US); Carlos A. Torres, Montgomery, TX (US); Ganesh Jagtap, Fayetteville, TX (US); Thomas E. Mueller, Fayetteville, TX (US)

(21) Appl. No.: 17/144,954

(22) Filed: Jan. 8, 2021

Related U.S. Application Data
(63) Continuation-in-part of application No. 16/961,068.
(60) Provisional application No. 62/959,610, filed on Jan. 10, 2020.
(60) Provisional application No. 62/621,282, filed on Jan. 24, 2018.

(51) Int. Cl. G16H 20/60; G16H 50/30

(52) U.S. Cl. CPC G16H 20/60; G16H 50/30

(57) ABSTRACT

An Application implemented on a computing device. Algorithms in the Application convert information about the nutrient concentration in a measure of food into a single quality rating number for the food. Concentration values of specific nutrients in the food are determined using information about the weight of selected nutrients in a measure of the food.

### CROSS REFERENCE TO RELATED APPLICATION

This application is a continuation-in-part of U.S. application Ser. No. 16/961,068 filed on Jul. 9, 2020, which in turn is a US national phase application of PCT/US2019/014963, filed Jan. 24, 2019, which claims priority to US 62/621,282, filed Jan. 24, 2018, all of the disclosures of which are incorporated herein by reference for all purposes. This application also claims priority to US 62/959,610, filed Jan. 10, 2020, the disclosure of which is incorporated herein by reference for all purposes.

### FIELD OF THE INVENTION

The present invention relates generally to rating food quality for guiding food selection and consumption.

### SETTING OF THE PRESENT INVENTION

The present invention addresses the recognized need for a simple assessment tool for applying dietary approaches appreciating that total amount of food is the driving force for satiety, and thus, the intake of low energy dense foods leads to a reduction in energy intake in obese subjects.

The identification and selection of foods that are best suited for individual health needs and objectives can be a daunting task. Most common grocery store foods carry a food label that lists ingredients and nutrient content. The food labels list contents for more than a dozen food attributes. Some, like dietary fiber, are very desirable, and others, like added sugar or saturated fats, should be avoided. Other important food attributes, like energy density and fiber density, are not even listed, leaving it up to the shopper to calculate the values. Trying to balance the good with the bad and choose what's right is a challenge even for people who have mastered the difficult technology behind nutrition and weight management.

Moreover, food labels do not address the personal situation of a shopper with specific weight management, nutrition, or health needs. Calorie and nutrient needs can vary widely depending on many factors that are unique to the individual. A common need is based on one's weight loss objective. However, not everyone needs to lose weight. Many need to maintain weight—especially those who have just completed a weight loss regimen. Studies show that up to 95% of dieters will shortly regain much of the weight they lost. Some need to gain weight. Some are under dietary restrictions regarding consumption of nutrients such as sodium and/or fat/cholesterol and/or other specific nutrients. Many people eat a dozen or more foods in a day, all with varying nutrient and energy attributes.

The present invention includes computing programs (Applications or Apps) having algorithms that calculate quality ratings for foods using information about the nutrient and energy content of the foods and the goals/nutritional requirements of the user. The algorithms used in calculating the density of nutrients in the food require knowledge about the weight of the food. Weight information for a food is typically included in the USDA databases and various commercial data sources. Weight information is not always available for multicomponent foods such as meals or other food items listed on a restaurant menu. Suppliers of multi-component foods frequently state the size of a serving portion in non-weight measurements such as pieces, cups, slices, servings, and other non-weight terms. The present invention teaches a method for approximating the weight of a food using information derived from the known nutrient content of the food. Calculations of food quality value numbers using the approximated weight closely track the values calculated using the total weight values of the foods.

### OBJECTS OF THE INVENTION

A primary object of the present invention is to simplify the identification and selection of foods that are best suited for individual health needs and objectives.

An object of the present invention is to provide an Application for use in a computing device, e.g., a computer, tablet, or cell phone, for calculating a single number that accurately rates a food's quality.

An important object of the present invention is to determine a value number rating the quality of a food using limited information about the food's nutrient content.

A related object of the present invention is to use a representative calculated weight of a food serving wherein the calculation is made using information about fewer than all the nutrients contained in the serving, and wherein such calculated weight value is used in algorithms that rate the food quality.

A related object of the present invention is to determine accurate representative values of nutrient densities in a quantity of food using weight values calculated from weight information about individual nutrients in the food.

Another object of the present invention is to provide a food quality rating method using information about the presence and density of individual nutrients in a quantity of food using a calculated total weight value that is accurately representative of such quantity of food.

### BRIEF DESCRIPTION OF DRAWINGS

FIG. 1 is a graph of the Statistical Correlation Using Known Food Weights and Calculated Food Weights.

### DESCRIPTION OF THE INVENTIONS

As used herein, the term “computer” refers to any computing device capable of running the Application described herein. Thus, computer includes but is not limited to a desktop, a laptop, a tablet, or a smart phone.

The task of identifying and selecting foods that are best suited for individual health needs and objectives is reduced to the calculation of a single number (One Number) that tells instantly whether any food is right or wrong for meeting those individual health needs and objectives. The One Number calculation system assigns all foods a number on a simple scale of 0 to 20. The best foods for the individual's situation have the highest numbers. Foods that are less healthy for the individual have lower numbers. In one implementation of the present invention, the user will effectively attain desired health needs and objectives by maintaining consumption of foods having a uniquely tailored One Number value of 10 or better. Because the system considers the specific nutritional needs and goals of the user, the same food can have a different One Number value for different users.

The base numerical component value of the rating number for a food item is calculated by summing the food item's protein content (in grams) and its fiber content (in grams) and dividing the sum by the number of calories in the serving portion of the food. In a preferred embodiment of the invention, a factor based on the value of the fiber density of the food is added to the base numerical component value to create the One Number rating value for a user having no dietary restrictions and wishing to maintain weight.

The base numerical component of the One Number rating value may be modified as a function of user dietary restrictions or weight management objectives. In the embodiments described and claimed herein, the One Number value is modified as a function of targeted nutrients such as the density of the sodium, fat and/or cholesterol in a serving. The density of any nutrient components in the serving portion is dependent on the individual weights of the nutrient components and the total weight of the serving portion. The density calculation for a nutrient is the weight of the nutrient in the serving portion divided by the serving portion weight.

In one form of the invention, algorithms are used to determine a base value for the One Number calculation using the food's content of protein, fiber, and calories. The algorithms further modify the base value as a function of the food's fiber density to determine a One Number value for a person wishing to have a healthy diet while maintaining weight. The base One Number value may be otherwise and/or further modified as a function of other targeted nutrients found in the food to address specific goals and objectives of the user.

Food for users with differing specific health or weight management concerns will have differing One Number food ratings. For example, boiled shrimp has a high One Number value of about 19 for the average healthy person wishing only to maintain weight. The One Number rating of boiled shrimp for a person who has a cholesterol concern is only about 4, a much lower value.

The computer and Application of the present invention are used to implement a healthy weight management plan and/or system (plan) for the individual user built around the One Number rating tool. The special needs of the user are programmed into the Application by targeting specific nutrients to formulate the weight management plan. The computer receives, processes, and displays the input information to produce a display and/or report showing the user's degree of compliance with the weight management plan.

The entry of the data may be accomplished through a barcode scanner, a quick response code (QR code) reader, a manual keyboard entry of the food's name, and/or a spoken name for the food. Databases containing nutrient information are carried in the computer memory and/or may also be accessed and supplied remotely through the internet or other electronic source. The nutrient information is processed by the Application in the computer and analyzed in conjunction with the user's weight management plan to provide current visual displays regarding attainment of the objectives and goals formulated in the weight management plan. The computer provides multiple, single-screen, real-time visual displays of the current One Number rating of the food, the quantity of selected nutrients consumed, the historical status of the aggregated cumulative quality of the food consumed, and the amount of body fat gained or lost during a monitored period.

Before being consumed, a food may be tested for its effect on body fat change, and the cumulative One Number rating value for food already consumed. Weight changes resulting from consuming that food are displayed as physical tablespoons of body fat to personalize and incentivize the user.

Recommended Daily Nutrient Allowances (RDA's) are tailored to the user and the specific weight management objectives. The visual displays also show such information as current values, historical status of the amounts of nutrients consumed, dietary balances and ratios, and net energy gain or deficit.

The foregoing description of the present invention may be more fully understood and appreciated by reference to U.S. Ser. No. 16/961,068 which is incorporated herein by reference for all purposes.

Estimating the Weight of a Serving of Food from Information Taken from its Ingredient Listing

The algorithms of the present invention calculate a single number (One Number) rating value for a food, based in part, on the density of specific nutrients in the food. This density is established by dividing the nutrient weight by the total weight of the food.

U.S. Ser. No. 16/961,068, incorporated herein by reference for all purposes, provides Equations 2.1 to 2.5 for calculation of the One Number rating value. These equations were created with correction factors (Fₓ) for use with databases and other information sources that provide the serving weight of the food being rated. The equations use the total weight of the food and calculate the concentration or density of cholesterol, fat/lipids, energy (kcal), fiber, and sodium. While the equations 2.1-2.5 rely on the total weight of the food, the total weight of the food is not always known or available. This is particularly so in the case of restaurant menu items where food quantity is frequently expressed in weightless terms such as “a serving”, “a piece”, “a slice”, or “a cup”. The densities of the nutrients in the food cannot be calculated using these weightless descriptions.

When the total weight of a food is not available, the algorithms of the invention calculate a representative food weight (Dry Weight) using the known nutrient weights of nutrient components of the food. One Number values calculated by the algorithms using the Dry Weight correlate closely with those calculated using the measured total weight of the food.

In a preferred form of the invention, an Application of the present invention is installed in a smart phone for calculating the One Number food quality ratings. The Application compiles information about ingredients in the food and the known value of a unit of weight of the nutrients themselves and calculates a Dry Weight value representative of the weight of the serving. The Dry Weight is then used in the calculation of the One Number quality rating for the food. The calculated value of the single number rating is modified to address the user's specific nutrition and health objectives.

Rating values calculated using the Dry Weight in the algorithms of the present invention are truncated below 0 and above 20. When used in these algorithms, the truncated Dry Weight rating values produce rating number values that accurately represent food quality specific to individual user goals and objectives.

Estimating Portion Weight from the Dry Weight

An important objective of the present invention is to calculate the various nutrient densities using a weight value determined from the known weight of the food nutrients in the food.

The weight of a portion of food equals the total of the weights of its ingredients, including water. The heaviest ingredients of most foods are water, protein, carbohydrates, and fats. The remaining nutrients in most foods contribute relatively little to the weight of the food item. The amount of water in the food has no bearing on the nutrient content.

The common food product label listing or restaurant menu food item listing of nutrients includes the portion size and the quantities of individual ingredients of total fat, cholesterol, sodium, total carbohydrate, protein, and various vitamins and minerals. Vitamins and minerals typically are listed in quantities of milligrams and micrograms. Total fat, total carbohydrates and protein content are typically listed in grams and usually account for the great majority of the total weight of the food.

Carbohydrates are defined by the USDA and FDA as anything besides water, alcohol, fat, protein, and ash. The weights of the carbohydrates, proteins, and fats in the product are reported and are thus known. Ash is a very nominal value, so it is not taken into consideration, leaving water and alcohol. Alcohol is also almost exclusively water. Alcohol is about 97% water. Carbohydrates have 4 calories per gram, protein has 4 calories per gram, and fat has 9 calories per gram.

The Dry Weight of the product is the sum of the weights of the protein+carbohydrates+fat. The densities of the individual nutrients in the product are calculated by dividing their weight by the Dry Weight.

The following list provides algorithms used in calculating the One Number rating value using the calculated Dry Weight rather than the total weight of the serving portion. For consistency and convenience of comparing food ratings, a program containing the algorithms of the present invention will preferably use the Dry Weight calculation method of the present invention to determine all One Number values for the food, regardless of whether the food item weight is known or not. As explained hereinafter, there is greater than a 90% correlation between the One Number values calculated using the total weight of the food portion and those calculated using the Dry Weight.

The list below describes the revised version of the equations 2.1-2.5 of the invention described in U.S. Pat. No. 16/961 068, incorporated herein by reference for all purposes. The revised version is used to calculate the One Number values using the calculated Dry Weight for the food.

### Dry Weight Equations 2.1d-2.5d

The revisions to the F corrections (Equations 2.1d-2.5d, given below) compared to the prior art Equations 2.1-2.5 listed in U.S. Pat. No. 16/961,068, include new coefficients in the numerator and a new version of weight in the denominator. Rather than dividing by the total weight of the serving of food, the equations divide by Dry Weight: the sum of Protein, Carbs, and Fat (all in g).

### Dry Weight=Protein+Carbs+Fat

F_{ch} is the concentration (or density) of cholesterol (in mg) in a food.

$${F}_{C h} = \frac{( C h \times . 018 \times 100 )}{Dry Weight} \qquad Equation 2.1 d$$

Thus, F_{ch} is computed as mg of cholesterol in a food times 0.018 times 100 divided by the dry weight (Protein+Carbs+Fat) of the food in grams.
F_{Fat} (also called F_{Lipids}) is the concentration (or density) of fat (in g) in a serving of food.

$${F}_{Fat} = \frac{( Fat \times .05 \times 100 )}{Dry Weight} \qquad Equation 2.2 d$$

Thus, F_{Fat} is computed as g of fat in a food times 0.05 times 100 divided by the dry weight (Protein+Carbs+Fat) of the food in grams.
F_{Energy} (also called F_{Eden}) is the concentration (or density) of energy (in kcal) in a serving of food.

$${F}_{Energy} = \frac{( Energy \times .0045 \times 100 )}{Dry Weight} \qquad Equation 2.3 d$$

Thus, F_{Energy} is computed as kcal of energy in a food times 0.0045 times 100 divided by the dry weight (Protein+Carbs+Fat) of the food in grams.
F_{Fiber} is the concentration (or density) of fiber (in g) in a serving of food.

$${F}_{Fiber} = \frac{( Fiber \times .09 \times 10 \; 0 )}{Dry Weight} \qquad Equation 2.4 d$$

Thus, F_{Fiber} is computed as g of fiber in a food times 0.09 times 100 divided by the dry weight (Protein+Carbs+Fat) of the food in grams.
F_{Na} is the concentration (or density) of sodium (in mg) in a food.

$${F}_{Na} = \frac{( Na \times .0057 \times 100 )}{Dry Weight} \qquad Equation 2.5 d$$

Thus, F_{Na} is computed as mg of sodium in a food times 0.0057 times 100 divided by the dry weight (Protein+Carbs+Fat) of the food in grams.

The correction factor coefficients in Equations 2.1d-2.5d provide factor coefficient values that produce One Number rating values that accurately rate food quality.

The quality difference between a food having a ranking of 14 and a food having a ranking of 20 may not be significant. The same may be said about two foods rated at the lower end of the scale. The food's One Number value informs the consumer as to the relative value of the food compared to foods having different rating values and lets the consumer know the relative healthiness of the food.

The calculated One Number rating values using the Dry Weight value are truncated at 0 and 20. The resulting OneNumber values closely approximate the number values obtained in the calculations using the actual total weight of the food. The truncation facilitates the implementation of the One Number rating system without compromising the significance of the rating number values within the 0-20 range.

When the serving portion weight of a meal or food item is not provided in the database, the following equations 2.1d-2.5d can be used to calculate a modified basic rating number using a calculated weight value (Dry Weight) representative of the total weight serving weight.

The following equations 2.6d-2.17d are used to calculate the One Number values using the Dry Weight calculations in equations 2.1d-2.5d for nutrient densities and meeting certain dietary and weight management goals.

### Category 1: Maintain Weight, No Dietary Restrictions (B4B₁)

$$B 4 {B}_{1} = \frac{g protein + ( 1.2 \cdot g fiber )}{100 calories} + {F}_{Fiber} \qquad Equation 2.6 d$$

### Category 2: Maintain Weight, Low Sodium Restriction (B4B₂)

$$B 4 {B}_{2} = \frac{g protein + ( 1.2 \cdot g fiber )}{100 calories} + {F}_{Fiber} - {F}_{Na} \qquad Equation 2.7 d$$

### Category 3: Maintain Weight, Low Fat, Low Cholesterol Restriction (B4B₃)

$$B 4 {B}_{3} = \frac{g protein + ( 1.2 \cdot g fiber )}{100 calories} + {F}_{Fiber} - {F}_{Ch} - {F}_{Fat} \qquad Equation 2.8 d$$

Category 4: Maintain Weight, Low Sodium, Low Fat, Low Cholesterol restriction (B4B₄)

$$B 4 {B}_{4} = \frac{g protein + ( 1.2 \cdot g fiber )}{100 calories} + {F}_{Fiber} - {F}_{Ch} - {F}_{Fat} - {F}_{Na} \qquad Equation 2.9 d$$

### Category 5: Lose Weight, No Dietary Restrictions (B4B₅)

$$B 4 {B}_{5} = \frac{g protein + ( 1.2 \cdot g fiber )}{100 calories} + {F}_{Fiber} - {F}_{Energy} \qquad Equation 2.10 d$$

### Category 6: Lose Weight, Low Sodium Restriction (B4B₆)

$$B 4 {B}_{6} = \frac{g protein + ( 1.2 \cdot g fiber )}{100 calories} + {F}_{Fiber} - {F}_{Energy} - {F}_{Na} \qquad Equation 2.11 d$$

### Category 7: Lose Weight, Low Fat, Low Cholesterol Restriction (B4B₇)

$$B 4 {B}_{7} = \frac{g protein + ( 1.2 \cdot g fiber )}{100 calories} + {F}_{Fiber} - {F}_{Energy} - {F}_{Ch} - {F}_{Fat} \qquad Equation 2.12 d$$

### Category 8: Lose Weight, Low Sodium, Low Fat, Low Cholesterol Restriction (B4B₈)

$$B 4 {B}_{8} = \frac{g protein + ( 1.2 \cdot g fiber )}{100 calories} + {F}_{Fiber} - {F}_{Energy} - {F}_{Ch} - {F}_{Na} - {F}_{Fat} \qquad Equation 2.13 d$$

### Category 9: Gain Weight, No Dietary Restrictions (B4₉)

$$B 4 {B}_{9} = \frac{g protein + ( 1.2 \cdot g fiber )}{100 calories} + {F}_{Fiber} ∓ {F}_{Energy} \qquad Equation 2.13 d$$

### Category 10: Gain Weight, Low Sodium Restriction (B4B₁₀)

$$B 4 {B}_{10} = \frac{g protein + ( 1.2 \cdot g fiber )}{100 calories} + {F}_{Fiber} + {F}_{Energy} - {F}_{N a} \qquad Equation 2.15 d$$

### Category 11: Gain Weight, Low Fat, Low Cholesterol Restriction (B4B₁₁)

$$B 4 {B}_{11} = \frac{g protein + ( 1.2 \cdot g fiber )}{100 calories} + {F}_{Fiber} + {F}_{Energy} - {F}_{Ch} - {F}_{Fat} \qquad Equation 2.16 d$$

Category 12: Gain Weight, low sodium, low fat, low cholesterol restriction (B4B₁₂)

$$B 4 {B}_{12} = \frac{g protein + ( 1.2 \cdot g fiber )}{100 calories} + {F}_{Fiber} + {F}_{Energy} - {F}_{Na} - {F}_{Ch} - {F}_{Fat} \qquad Equation 2.17 d$$

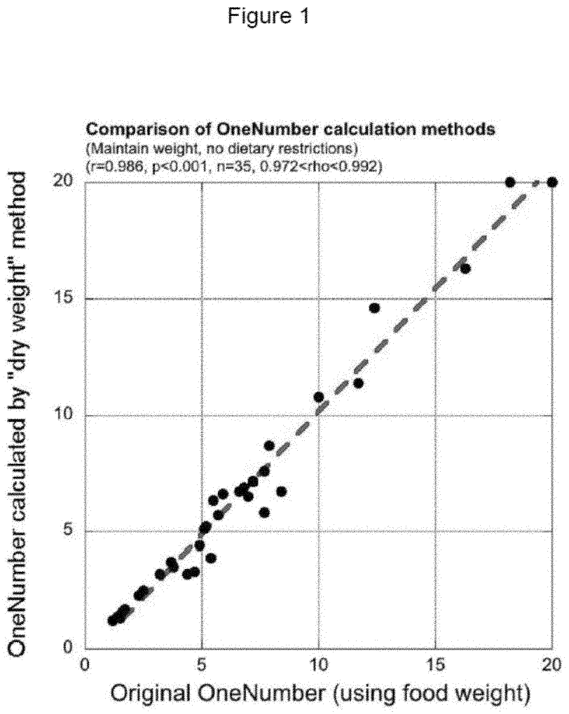

Validation of the Dry Weight nutrient density algorithms calculations is illustrated by confirmation that the One Number values calculated for listings of foods using the Dry Weight nutrient density are ranked in substantially the same order as the One Number values calculated using the total weight of the serving. The One Number formula modification values calculated using total weight and those calculated using the Dry Weight have been tested sufficiently to confirm a statistical correlation greater than 90%. See FIG. 1 which is a graph of the Statistical Correlation Using Known Food Weights and Calculated Food Weights.

1. A method of calculating a food value rating number for a food item containing multiple nutrient components and an energy component for determining the suitability of the food item in meeting an individual's healthy weight management plan comprising:
determining the weights of selected nutrients in the food item;
combining the weights of the selected nutrients in the food item to obtain a dry weight value representative of the total weight of the food item;
determining a nutrient density value for the selected nutrients using the dry weight value;
determining the energy value of the food item;
determining an energy density of one or more of said selected nutrients using the energy value of the food item; and
determining a base rating value number for the food item using a ratio value derived from the energy density of one or more of said selected nutrients.

2. The method as defined in claim 1 wherein said base rating value number is determined by the energy density of the protein and fiber in the food.

3. The method as defined in claim 2 wherein said ratio value is modified as a function of the density of at least one of the selected nutrients.

4. The method as defined in claim 3 wherein said ratio value is modified as a function of the density of the fiber in the food.

5. The method as defined in claim 4 wherein said ratio value is modified as a function of a food nutrient targeted in said individual's plan.

6. The method as defined in claim 1, further comprising:
truncating said base rating value number below 0 and above 20 such that said base rating value number is from 0 to 20, inclusive of 0 and 20.

7. The method as defined in claim 1 wherein said base food rating value rating number is used as a part of a computer application to rate the nutritional quality of food.

8. The method as defined in claim 1 wherein said rating number value is raised or lowered as a function of one or more specific nutrients in the food.

9. The method as defined in claim 1 wherein said base rating number is increased in value as a function of the food's fiber density.

10. The method as defined in claim 1, wherein said food value rating number is calculated by the following equations according to the specific objectives or needs of the individual, wherein
(a) for an individual who seeks to maintain weight with no dietary restrictions, the following equation is used:
$$B 4 {B}_{1} = \frac{g protein + ( 1.2 \cdot g fiber )}{100 calories} + {F}_{Fiber} \qquad Equation 2.6 d$$
(b) for an individual who seeks to maintain weight with a low sodium restriction, the following equation is used:
$$B 4 {B}_{2} = \frac{g protein + ( 1.2 \cdot g fiber )}{100 calories} + {F}_{Fiber} - {F}_{Na} \qquad Equation 2.7 d$$
(c) for an individual who seeks to maintain weight with low fat and low cholesterol restrictions, the following equation is used:
$${B 4 B}_{3} = \frac{g protein + ( 1.2 \cdot g fiber )}{100 calories} + {F}_{Fiber} - {F}_{Ch} - {F}_{Fat} \qquad Equation 2.8 d$$
(d) for an individual who seeks to maintain weight with low sodium, low fat, and low cholesterol restrictions, the following equation is used:
$${B 4 B}_{4} = \frac{g protein + ( 1.2 \cdot g fiber )}{100 calories} + {F}_{Fiber} - {F}_{Ch} - {F}_{Fat} - {F}_{Na} \qquad Equation 2.9 d$$
(e) for an individual who seeks to lose weight with no dietary restrictions, the following equation is used:
$${B 4 B}_{5} = \frac{g protein + ( 1.2 \cdot g fiber )}{100 calories} + {F}_{Fiber} - {F}_{Energy} \qquad Equation 2.10 d$$
(f) for an individual who seeks to lose weight with low sodium restrictions, the following equation is used:
$${B 4 B}_{6} = \frac{g protein + ( 1.2 \cdot g fiber )}{100 calories} + {F}_{Fiber} - {F}_{Energy} - {F}_{Na} \qquad Equation 2.11 d$$
(g) for an individual who seeks to lose weight with low fat and low cholesterol restrictions, the following equation is used:
$${B 4 B}_{7} = \frac{g protein + ( 1.2 \cdot g fiber )}{100 calories} + {F}_{Fiber} - {F}_{Energy} - {F}_{Ch} - {F}_{Fat} \qquad Equation 2.12 d$$
(h) for an individual who seeks to lose weight with low sodium, low fat, and low cholesterol restrictions, the following equation is used:
$${B 4 B}_{8} = \frac{g protein + ( 1.2 \cdot g fiber )}{100 calories} + {F}_{Fiber} - {F}_{Energy} - {F}_{Ch} - {F}_{Na} - {F}_{Fat} \qquad Equation 2.13 d$$
(i) for an individual who seeks to gain weight with no dietary restrictions, the following equation is used:
$${B 4 B}_{9} = \frac{g protein + ( 1.2 \cdot g fiber )}{100 calories} + {F}_{Fiber} + {F}_{Energy} \qquad Equation 2.14 d$$
(j) for an individual who seeks to gain weight with low sodium restrictions, the following equation is used:
$$B 4 {B}_{10} = \frac{g protein + ( 1.2 \cdot g fiber )}{100 calories} + {F}_{Fiber} + {F}_{Energy} - {F}_{N a} \qquad Equation 2.15 d$$
(k) for an individual who seeks to gain weight with low fat and low cholesterol restrictions, the following equation is used:
$$B 4 {B}_{11} = \frac{g protein + ( 1.2 \cdot g fiber )}{100 calories} + {F}_{Fiber} + {F}_{Energy} - {F}_{Ch} - {F}_{Fat} \qquad Equation 2.16 d$$
(l) for an individual who seeks to gain weight with low sodium, low fat, and low cholesterol restrictions, the following equation is used:
$$B 4 {B}_{12} = \frac{g protein + ( 1.2 \cdot g fiber )}{100 calories} + {F}_{Fiber} + {F}_{Energy} - {F}_{Na} - {F}_{Ch} - {F}_{Fat} \qquad Equation 2.17 d$$
